(19) United States
(12) Patent Application Publication
(10) Pub. No.: US 2009/0023638 A1
(43) Pub. Date: Jan. 22, 2009

(54) SPILL RESISTANT ANTIBIOTIC FORMULATIONS

(75) Inventors: Satish Asotra, Brampton (CA); Shen Gao, Bolton (CA)

Correspondence Address: TARO PHARMACEUTICALS U.S.A., INC.;C/O VENABLE LLP, P.O. BOX 34385, WASHINGTON, DC, 20043-9998, US

(73) Assignee: Taro Pharmaceuticals North American, Inc., Grand Cayman (KY)

(21) Appl. No.: 12/162,675

(22) PCT Filed: Feb. 2, 2007
(86) PCT No.: PCT/US07/02910

§ 371 Date: Jul. 30, 2008

Related U.S. Application Data
(60) Provisional application No. 60/765,614, filed on Feb. 6, 2006.

(51) Int. Cl. A61K 38/14; A61K 31/43; A61K 31/546; A61K 31/7052; A61K 31/7048

(52) U.S. Cl. 514/8; 514/192; 514/202; 514/29

(57) ABSTRACT

The disclosure is directed to a pharmaceutical kit with a first set, a second set and a third set of components. The first set of components can be a carbomer in an aqueous vehicle, the second set of components can be an antibiotic, and the third set of components can be a neutralizing agent. In exemplary embodiments, the first, second and third sets of components can be mixed together to form a spill resistant composition.

### BACKGROUND

Patient compliance with medical advice concerning medications is a particular problem in the clinical practice of medicine. Non-compliance with medication instructions can lead to treatment failure, deterioration of the patient's health, additional consultations and increases in the direct and indirect cost of health care. Even in cases of acute disease, where the patient motivation to comply should be high, a significant percentage of patients are not compliant. Compliance with a dosing regime is associated with the convenience of therapy. Especially in the pediatric population, the provider's impression of the ease or difficulty of administering a drug is associated with increased compliance. In addition to compliance, the issue of incomplete dosing is a particular problem with liquid, oral formulations Inaccurate measurement devices, overfilling or underfilling of the device, insufficient uniformity of product due to lack of shaking before administration, and spillage of the product during administration from bottle to patient, all contribute to inaccurate dosage of the patient.

Antibiotics are commonly given for the treatment or prevention of bacterial diseases. In many cases, the antibiotics are given for a short course of treatment of ten days or less. Tablets, capsules and liquid formulations are common dosage forms for antibiotic administration. For the pediatric composition, the antibiotics are often supplied in dry powder form and reconstituted with water for patient administration. The resultant solution or suspension is in a liquid form Compliance and accurate dosing are particularly relevant to antibiotic administration because in addition to deterioration of patient health, increased visits to health care providers, loss of work and general increase in costs and resources, incomplete compliance with medical instructions may contribute to the proliferation of drug resistant strains of antibiotics.

Spill-resistant pharmaceutical formulations have been described in U.S. Pat. No. 6,102,254, U.S. Pat. No. 6,071,523 and U.S. Pat. No. 6,399,079, herein incorporated by reference. The spill resistant formulations have been described by the physical properties of (a) extrudability under light manual pressure from a squeezable container or a proxy (e.g. a syringe with a 5 mm orifice), and (b) spreadability in a spoon bowl measured by extruding the formulation into a spoon bowl and determining whether the material levels or spreads to the edges of the spoon bowl. The extrudability and spreadability of the spill resistant formulation allows for ease of administration. Accuracy of measurement and the homogenous dispersion of the active pharmaceutical ingredient in the spill resistant solution ensure accuracy of delivery of the pharmaceutical product.

Inventive containers are necessary for the delivery of spill resistant pharmaceutical formulations because the base components will not pour easily from non-squeezable containers. Containers for spill resistant pharmaceutical formulations have been disclosed and claimed in U.S. Pat. No. 6,745,919, herein incorporated by reference.

A problem with the spill resistant formulations has been the need to prepare shelf storage compositions that retain the active ingredient and other components in solution or suspension without degradation of the spill resistance properties for periods of up to two years. We describe herein, an easy to prepare and use kit for the preparation and delivery of antibiotic liquid formulations to patients in need of safe and accurate delivery system of antibiotics.

### SUMMARY OF THE INVENTION

It is an object of this invention to provide a pharmaceutical kit comprising a first set, a second set and a third set of components. The first set of components comprises a carbomer in an aqueous vehicle, the second set of components comprises an antibiotic or mixes thereof and the third set contains a neutralizing agent wherein when said first, second and third sets of components when mixed together form a spill resistant composition having a pH of about 4.6 to about 7.0.

The kit of may have antibiotics selected from the group consisting of drugs from cephalasporins, beta-lactamase inhibitors, monobactams, beta-lactams, quinolones, macrolides, aminoglycosides, vancomycin, streptogramins oxazolindinones, alone or in combination.

In one aspect of the invention, the kit contains an antibiotic is selected from the group consisting of drugs from amoxicillin, cefdinir, azithromycin, erythromycin, clairthromycin, ciprofloxacin, cephalexin and combinations thereof.

The spill resistant properties of the may include the aqueous vehicle in a squeezable container that is free of indentations and is smooth sided.

The aqueous vehicle component of the kit may comprise sucralose, sorbitol, a carbomer, glycerin, propylene glycol, butyl paraben and other pharmaceutically acceptable excipients.

The invention also provides for a process for preparing a spill resistant pharmaceutical composition comprising an antibiotic, a base component and a neutralizing solution wherein the process comprises the steps of adding the antibiotic to the base component to form a suspension or a solution and adding a neutralizing solution to a pH of between about pH of about 4.6 to about 7.0.

### DETAILED DESCRIPTION

In describing embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose. The above-described embodiments of the invention may be modified or varied, and elements added or omitted, without departing from the invention, as appreciated by those skilled in the art in light of the above teachings. Each reference cited here is incorporated by reference as if each were individually incorporated by reference.

The present invention comprises a kit containing three components. Component A of the present invention is a diluent and is provided in a container. The diluent is comprised of a thickener and a carrier, and may include organoleptic components in an aqueous solution. Carbomers (Merck Index 12^{th} ed., no. 1878) can be used as thickeners in semi-solid pharmaceutical formulations (see Mehta et al., U.S. Pat. No. 6,071,523). Carbomer 934P (Carbopol® 974P) is a suitable thickener or gelling agent. Suitable concentrations from about 0.01 to about 1.0%, and more specifically from about 0.40 to about 0.45%, w/w. Carbomers rheology supports a high yield value (Handbook of Pharmaceutical Excipients Third Ed., A. H. Kibbe (Ed.), Pharmaceutical Press, London, UK, 2000, Pg. 442, 79, 53 (“Handbook of Pharm. Excipients”). Carbomers are slightly acidic and must be neutralized e.g. with sodium hydroxide (as needed to neutralize the carbomer up to about 0.08% in particular formulations) with a preferred pH range being about 6.2 to about 7.3, providing the maximum viscosity plateau. The vehicle carrier component comprises propylene glycol up to about 20%, or from about 3 to about 10%. Glycerin up to about 50% may be present. Additionally, sorbitol, up to 10%, may be added as a vehicle and stabilizer. Purified water comprises the bulk of the carrier component comprising from about 29% to about 64% of the formulation.

The pharmaceutical solutions, and suspensions may comprise at least one additional component selected from the group consisting of excipients, surface active agents, dispersing agents, inert diluents, granulating agents, disintegrating agents, binding agents, lubricating agents, sweetening agents, flavoring agents, coloring agents, preservatives, oily vehicles, solvents, suspending agents, dispersing agents, wetting agents, emulsifying agents, demulcents, buffers, salts, fillers, antioxidants, antibiotics; antifungal agents and stabilizing agents.

The organoleptic ingredients improve the taste and appearance and do not negatively affect the solution, stability. The organoleptic agents in the following examples include coloring and flavoring agents, sweeteners and masking agents.

The diluent is contained within a dispenser that is unique to the kit. In a particular embodiment, the squeezable container has no shoulders, and no inversions of the mold, and thus offers ease of mixing and ensures that a homogenous distribution of the active pharmaceutical ingredient in the finished drug product.

Component B is a pharmaceutically active ingredient of the antibiotic class of drugs. It is anticipated that any antibiotic may have utility in the invention. The antibiotic may be selected from the group consisting of drugs from cephalasporins, beta-lactamase inhibitors, monobactams, beta-lactams, quinolones, macrolides, aminoglycosides, vancomycin, streptogramins oxazolindinones, alone or in combination. Specific examples of antibiotics to be used with the invention may be amoxicillin, cefdinir, azithromycin, erythromycin, clairthromycin, ciprofloxacin, cephalexin and combinations thereof. The antibiotic may be in the solid state as a dry powder, or in a liquid medium. The antibiotic powder of component B may be the base or salt form of the active ingredient. The powder may contain coated or uncoated particles of the antibiotic. The particles of the dry antibiotic composition remain stable for about 48 months without significant decomposition.

Component B may additionally comprise adispersant, a surfactant or wetting agent as excipients. In producing such a therapeutic composition according to the present invention, the surfactant, and the dispersant may be present with and in combination with the antibiotic or the like particles in any suitable combination and the inclusion of any one or more of these adjuncts would fall within the scope of this invention and would serve to some degree to promote a more rapid dissolution or suspension of the antibiotic ingested and utilized as described herein. The surfactants may be selected from any one of several types of surfactants, any of which promote the wetting and dissolution of antibiotic particles. Examples of such surfactants may include the polyoxypropylene-polyoxyethylene nonionics; the alkyl phenoxy poly(ethyleneoxy) ethanols or ethoxylated alkyl phenols; the ethoxylated aliphatic alcohols or alkyl poly(ethyleneoxy) ethanols; the organic phosphoric acid esters the polyoxyethylene derivatives of sorbitan fatty acid esters; lecithin; and others. These have all been found to be beneficial toward the end of wetting and dissolving of the antibiotics component.

The term “spill resistant formulation” refers here to a product which, as sold, has viscosity in a certain range (e.g. 5,000 to 15,000 cps), is a semi-solid, is easy to administer accurately, has spill-resistant consistency, is storage stable, and has mutually compatible ingredients, as described in Mehta et al., U.S. Pat. No. 6,071,523, herein incorporated by reference. Viscosity can be measured using a Brookfield Viscometer with a ‘T-C’ spindle at 20 RPM and 20-25 degrees C., or equivalent. Viscosity decreases slightly with increasing temperature.

The invention relates to a pharmaceutical formulation for oral administration, comprising an effective amount of particles of a water soluble or water insoluble antibiotic in a pharmaceutically acceptable aqueous vehicle. The inventive formulation have some or all of the following qualities. First, the suspension may have a homogeneity wherein the antibiotic is uniformly dispersed or dissolved in the vehicle. The formulation also may have a stability such that the active ingredient remains indefinitely without agitation, that is without stirring or =shaking. The antibiotic remains uniform per dose administered, and does not fall out of solution. A semi-solid formulation of the invention can not be shaken easily, so the active agent must remain suspended or in solution without shaking. Advantageously, there is no need to shake the inventive compositions. The formulation may have a spill-resistant consistency permitting the composition to be squeezed into a spoon from a container with light manual pressure, to spread and level in a spoon bowl quickly enough for accurate measurement (typically in about 1-5 seconds at room temperature), and to remain in the spoon bowl long enough to permit administration without spilling particularly under difficult circumstances such as encountered with dispensing to children, or by the elderly. Spill-resistance refers to the product's ability to withstand a series of tests that were developed to evaluate the product's spill resistance. For most formulations, spill resistance means the formulation does not spill from a teaspoon for a definite period, e.g. at least about 30 or 60 seconds on spoon inversion, up to about 60 seconds on spoon vibration, and about 10, 20, or 30 seconds on spoon tilting. Spill resistant properties correlate with viscosity but are not necessarily directly linked, so that a composition within the target viscosity range may lack spill resistance. The shaking, tilting and inversion tests are performed on an experimental platform as described in U.S. Pat. No. 6,071,523. Spill resistance is related to whether the formulation passes a flow test, ensuring that dispensing and dosing to a 5.0 mL teaspoon is easy and satisfactorily accurate.

The formulation may have a flow quality having a non-Newtonian, pseudoplastic and time independent fluidity wherein the viscosity of the non-solid gel decreases with increasing shear rate, in which the behavior is fully reversible, and is indicative of Bingham behavior. The inventive spill resistant formulations are non-Newtonian and time independent fluids. Non-Newtonian refers to a fluid whose behavior departs from that of an ideal Newtonian fluid. These fluids have different viscosities at different shear rates and fall under two groups: time independent and time dependent. In contrast, for a Newtonian fluid the rate of shear in the fluid under isothermal conditions is proportional to the corresponding stress at the point under consideration. Time independent fluids are those for which the rate of shear at any point in the fluid is some function of the shear stress at that point and depends on nothing else. These fluids have a constant viscosity value at a given shear rate. The viscosities do not change with time. (McGraw-Hill Encyclopedia of Science & Technology, 6^{th} edition, 1987, Volume 12, pages 57-60).

Pharmaceutical formulations according to the invention comprise an antibiotic or a pharmaceutically acceptable salt or ester thereof as an active ingredient together with one or more pharmaceutically acceptable carriers, excipients or diluents. The inventive formulations have attractive appearance, suitable texture and organoleptic properties. The components are mutually compatible in that they do not interfere with the bioactivity of the pharmaceutical agent or physical properties of the vehicle, and the components do not separate and retain their properties.

### EXAMPLES

### Spill Resistant Reconstituted Antibiotics

### Example 1

### Component A—The Diluent

The following ingredients were combined and stored in a polyethylene container.

| | Amount |
|---|---|
| Ingredient | (w/w %) |
| Purified Water | 33.8 |
| Sucralose Liquid Concentrate | 0.30 |
| Sorbitol crystalline | 5.0 |
| Carbomer 934P (Carbopol 974P) | 0.48 |
| Glycerin | 50 |
| Propylene Glycol | 10 |
| Butyl Paraben | 0.04 |
| Masking agent | 0.30 |

### Example 2

### Preparation of Reconstituted Spill Resistant Compositions

i. Azithromycin Monohydrate 100 mg/5 mL; 200 mL/5 mL

1.82 or 3.64 grams of azithromycin monohydrate (Taro Pharmaceuticals, Ltd.) was added to the 100 ml of the component A, and vigorously shaken. 0.30 ml of a 10% solution of sodium hydroxide was added to bring to the final pH e to ensure spill resistant properties of the product. This pH will also warrant chemical stability of the spill resistant product.

The resultant suspensions were found to meet the shaking, spilling and filling test of a spill resistant formulation.

Erythromycin 125 mg/5 mL

2.27 grams of erythromycin (Alembic Limited, Gujarat, India) was added to the 100 ml of the component A, and vigorously shaken. 0.30 ml of a 10% solution of sodium hydroxide was added to bring to a final pH. This pH will also warrant chemical stability of the spill resistant product.

The resultant suspensions were found to meet the shaking, spilling and filling test of a spill resistant formulation.

Claritromycin 114 mg/5 mL

2.28 grams of clarithromycin (Matrix Laboratories Limited, Secunderabad, India) was added to the 100 ml of the component A, and vigorously shaken. 0.30 ml of a 10% solution of sodium hydroxide was added to bring to a final pH. This pH will also warrant chemical stability of the spill resistant product. The resultant suspensions were found to meet the shaking, spilling and filling test of a spill resistant formulation.

### Other Examples

Using the above procedures with commercially available Amoxicillin, Augmentin and Omnicef dry products, spill resistant versions were successfully achieved.

1. A pharmaceutical kit comprising a first set, a second set and a third set of components, wherein said first set of components comprises a carbomer in an aqueous vehicle, said second set of components comprises an antibiotic, said third set contains a neutralizing agent wherein when said first, second and third sets of components when mixed together form a spill resistant composition having a pH of about 4.6 to about 7.0.

2. The kit of claim 1 wherein the antibiotic is selected from the group consisting of drugs from cephalasporins, beta-lactamase inhibitors, monobactams, beta-lactams, quinolones, macrolides, aminoglycosides, vancomycin, streptogramins oxazolindinones, alone or in combination.

3. The kit of claim 1 wherein the antibiotic is selected from the group consisting of drugs from amoxicillin, cefdinir, azithromycin, erythromycin, clairthromycin, ciprofloxacin, cephalexin and combinations thereof.

4. The kit of claim 1 wherein the first set of components in a squeezable container that is free of indentations and is smooth sided.

5. The kit of claim 1 wherein the first set of components is an aqueous vehicle comprising, sucralose, sorbitol, a carbomer, glycerin, propylene glycol and butyl paraben.

6. A process for preparing a spill resistant pharmaceutical composition comprising an antibiotic, a base component and a neutralizing solution wherein the process comprises the steps of adding the antibiotic to the base component to form a suspension or a solution and adding a neutralizing solution to a pH of between about pH of about 4.6 to about 7.0.
